(19) United States
(12) Patent Application Publication
(10) Pub. No.: US 2017/0065234 A1
(43) Pub. Date: Mar. 9, 2017

(54) Method, Computer Program, and System for Determining the Spatial Course of a Body, in Particular of an Electrode, on the Basis of at Least a 2D X-Ray Image of the Electrode

(71) Applicant: BIOTRONIK SE & Co. KG, Berlin (DE)

(72) Inventor: Jens Rump, Berlin (DE)

(21) Appl. No.: 15/221,058

(22) Filed: Jul. 27, 2016

(30) Foreign Application Priority Data
Sep. 8, 2015 (DE) 10 2015 115 060.3

(51) Int. Cl. A61B 6/12; A61B 6/00; A61B 90/00

(52) U.S. Cl. CPC A61B 6/12; A61B 90/39; A61B 6/5217; A61B 2090/3966

(57) ABSTRACT

A method for reconstructing the spatial course of an elongate, flexible in a 3D world coordinate system, wherein the body has a plurality of x-ray markers, which are arranged on the body distanced from one another along said body, comprising: providing a 2D x-ray image of the body; determining the two-dimensional positions of the x-ray markers in an image coordinate system of the 2D x-ray image; determining possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane; and repeatedly determining the spatial course of the body with use of said possible 3D location coordinates. A corresponding computer program and a corresponding system are also provided.

### CROSS-REFERENCE TO RELATED APPLICATIONS

This patent application claims the benefit of and priority to co-pending German Patent Application No. DE 10 2015 115 060.3, filed on Sep. 8, 2015 in the German Patent Office, which is hereby incorporated by reference in its entirety.

### TECHNICAL FIELD

The present invention relates to a method, a computer program, and a system for reconstructing the spatial course of an elongate flexible electrode in a 3-dimensional (“3D”) world coordinate system.

### BACKGROUND

What is key to the successful implantation of electrodes (for example, for cardiac pacemakers) is the optimal placement of the electrode within the heart. For this reason, the implantation is generally monitored by recorded real-time fluoroscopy images obtained by means of x-ray imaging. With these recorded images, however, the electrode position within the heart can be ascertained as a radioscopic image only from a single view. In order to check the electrode position from a different viewing angle, the imaging apparatus has to be re-adjusted. Apparatuses that at the same time enable imaging from more than one viewing direction (e.g., biplanar radiography, CT, MRI) are often not available or are unsuitable for inter-operative imaging. In addition, in the case of biplanar radiography or CT, the radiation exposure for the doctor and patient increases significantly. Furthermore, active 3D tracking methods are often costly and usually only available in the research field.

The present invention is directed toward overcoming one or more of the above-mentioned problems.

### SUMMARY

On this basis, the object of the present invention is to provide a method, a computer program, and a cistern for reconstructing the spatial course of a body, in particular an electrode, which overcomes the aforementioned disadvantages, at least in part.

At least this problem is solved by a method according to claim 1, a computer program according to claim 6, and by a system according to claim 9.

Advantageous embodiments of these aspects of the present invention are specified in the associated dependent claims and will be described hereinafter.

In accordance with claim 1, a method for reconstructing the spatial course of an elongate, flexible body in a 3D world coordinate system is provided, wherein the body has a plurality of x-ray markers, which are arranged on the body distanced from one another along said body, and wherein the method has the following steps:
providing a 2-dimensional (“2D”) x-ray image of the body,
determining the two-dimensional positions of the x-ray markers in a 2D image coordinate system of the 2D x-ray image,
determining possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane of the x-ray image, and
repeatedly determining the spatial course of the body (for example, in the form of a fitted 3D curve) with use of said possible 3D location coordinates.

Here, the term x-ray marker means that these markers are sufficiently impermeable to x-ray beams, such that they form a detectable contrast in an x-ray image.

In other words, the present invention thus makes it possible, as a result of a uniform arrangement of x-ray markers along the body or the electrode, to obtain additional information regarding the 3D position of the body or of the electrode, more specifically regarding the projected geometry of the markers, such that it is possible to reconstruct the 3D position of the body or of the electrode on the basis solely of a 2D imaging.

In accordance with one embodiment of the method according to the present invention, provision is made—as already mentioned—for the body to have an electrode or to be formed as an electrode. This is preferably flexible, that is to say bendable, and is preferably elongate. The latter means that the body or the electrode, along a longitudinal axis along which said body or electrode extends from a distal end to a proximal end, has a greater extension than perpendicularly to the longitudinal axis.

In accordance with a preferred embodiment of the method according to the present invention, provision is also made for said repeated determination to comprise the following steps:
(a) preferably pre-defining starting values for the 3D location coordinates from the set of possible 3D location coordinates (for example, in a plane centrally between the radiation source and a detector or the image coordinate plane parallel to the detector or the image coordinate plane, more specifically one point in the plane per x-ray marker) and fitting a 3D curve to the initial 3D location coordinates, thus obtained, of the x-ray markers in the world coordinate system,
(b) shifting at least one 3D location coordinate along the associated beam to a possible further 3D location coordinate in order to obtain updated 3D location coordinates,
(c) fitting a 3D curve to the updated 3D location coordinates,
(d) back-projecting the 3D curve into the image coordinate system (onto the 2D image coordinates) and comparing the back-projection with the 2D x-ray image, in particular in terms of the size, orientation and/or position of the x-ray markers in the x-ray image,
(e) continuing the repeated determination starting with step (b) until a predefined criterion is reached.

Following a comparison between this projection and the actual position of the electrode in the x-ray image, the location position (3D location coordinates) of the markers is preferably modified accordingly, and the most likely position is determined by conventional optimization algorithms (for example, gradient methods, particle swarm optimization, genetic algorithm). The termination criteria for the optimization are given from the sought accuracy of the reconstruction. Here, the theoretical minimum of the optimization is a difference from the back-projection and 2D x-ray image that lies in the range of the image resolution of the x-ray image. Here, it must be taken into consideration that the duration of the optimization process increases with increased sought accuracy.

The target variable or the target variables minimized by the optimization is/are preferably the deviations of the back-projection of the individual components of the object or sleeves from the 2D x-ray image. If, compared with the 2D x-ray image, the back-projection is too large, the location coordinates are preferably shifted in the direction of the image coordinate plane. If they are too small, they are preferably shifted in the direction of the radiation sources. The exact system in accordance with which the coordinates of the 3D location coordinates are shifted can be dependent on the selected algorithm of the optimization or is determined by the selected optimization method (see above).

Since the distance between adjacent sleeves is clearly defined (this is based on a linearly extended body) and the maximum curvature can be limited on account of the elastic properties and the diameter of the body or of the electrode, a spherical shell is provided, for each possible position of a sleeve n on the corresponding straight line or the corresponding beam, for the possible position of the adjacent sleeve n+1.

In accordance with a preferred embodiment of the method according to the present invention, provision is therefore also made for the further location coordinate, in the above-discussed step (b), to be selected such that it lies on the beam associated with the further location coordinate and in a spherical shell around a 3D location coordinate of an adjacent x-ray marker, wherein the outer radius of the spherical shell is given by the distance between the two adjacent x-ray markers along the body, and wherein the inner radius of the spherical shell is given by the length of a chord extended between the two x-ray markers with maximum curvature of the body between the two adjacent x-ray markers.

The outer radius is preferably equal to the distance between the adjacent sleeves in question: Router=D, which is based on a linear course of the body.

The inner radius of the spherical shell is preferably given here by the length of the chord with maximum curvature r: Rinner=2*r*sin(D/(2*r)).

The straight-line portions or beam portions given from the intersection of the spherical shell with the beams of the x-ray marker n+1 limit the possible 3D positions of the markers for the optimization of the position of the body or electrode to at most two regions within the spherical shell.

The information concerning which of the two regions comes into question for the position of the sleeve can be determined from the parallactic enlargement of the sleeve. Additional information is provided by the angular position and geometric shortening of the marker in question or the sleeve in question in the detector plane or the image coordinate plane. The spatial resolution capability outside the image axis is primarily dependent on the resolution capability of the x-ray image.

In accordance with a preferred embodiment of the method according to the present invention, provision is also made for the x-ray markers to be annular, more specifically formed in particular as sleeves, wherein these sleeves can be metal sleeves, for example.

In accordance with a preferred embodiment of the method according to the present invention, provision is also made for adjacent x-ray markers to be arranged at distances from one another of 1 cm to 5 cm, preferably 2 cm. In particular, the (for example, metal) sleeve can have an outer diameter of 2 mm and a length of 1 mm along the longitudinal axis of the body or of the electrode. The distance 2 cm is given in particular, for example, from the assumption that average radii of curvature of the electrodes in the intracardial field are approximately r=2 cm (curvature: 0.5 l/cm). The wall thickness of the sleeves should be sufficient for a visible x-ray contrast and is greater than or equal to 0.05 mm in accordance with one embodiment.

Furthermore, in accordance with a preferred embodiment of the method according to the present invention, provision is made for the x-ray markers to comprise metal particles, which are introduced in ring form into an insulation of the body or electrode.

In accordance with a preferred embodiment of the method according to the present invention, provision is also made for the x-ray markers to have a metal braid, which is arranged in an insulation of the body or electrode.

In accordance with a preferred embodiment of the method according to the present invention, provision is also made for the x-ray markers to differ from one another in terms of their spatial dimensions, in particular depending on their position along the body or the electrode, in particular in such a way that the corresponding contrasts of the x-ray markers in the 2D x-ray image can be distinguished from one another.

In accordance with a further aspect of the present invention, a computer program for reconstructing the spatial course of an elongate, flexible body in a 3D world coordinate system is disclosed, wherein the body has a plurality of x-ray markers, which are arranged on the body at a distance from one another along said body, and wherein the computer program has a program code, which is configured to perform the following steps when the computer program is run on a computer:
determining the two-dimensional positions of the x-ray markers in a 2D image coordinate system of a 2D x-ray image recorded by the body,
determining possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane, and
repeatedly determining the spatial course of the body with use of said possible 3D location coordinates.

In accordance with a preferred embodiment of the computer program according to the present invention, provision is again made for the body to comprise an electrode or to be formed as an electrode (see above as well).

In accordance with one embodiment of the computer program according to the present invention, provision is again also made for said repeated determination to comprise the following steps:
(a) fitting a 3D curve to predefined starting values for the 3D location coordinates of the x-ray markers (world coordinate system) from the set of possible 3D location coordinates,
(b) shifting at least one 3D location coordinate of the 3D curve along the associated beam to a possible further 3D location coordinate,
(c) fitting a 3D curve to the 3D location coordinates of the 3D curve updated in this way,
(d) back-projecting this 3D curve into the image coordinate system (onto the 2D image coordinates) and comparing the back-projection with the 2D x-ray image, in particular in terms of the size, orientation and/or position of the x-ray markers in the x-ray image,
(e) continuing the repeated determination starting with step (b) until a predefined criterion is reached.

Reference is made in this regard to the explanations above.

In accordance with a preferred embodiment of the computer program according to the present invention, the further location coordinates is again, as already presented above, selected in step (b) such that it lies on the beam associated with the further location coordinate and in a spherical shell around a 3D location coordinate of an adjacent x-ray marker, wherein the outer radius of the spherical shell is given by the distance between the two adjacent x-ray markers along the body, and wherein the inner radius of the spherical shell is given by the length of a chord extended between the two x-ray markers with maximum curvature of the body between the two adjacent x-ray markers.

The x-ray markers are again preferably formed and arranged relative to one another in one of the ways already described above.

In accordance with a further aspect of the present invention, a system for reconstructing the spatial course of an elongate flexible body in a 3D world coordinate system is proposed, wherein the system has at least: an elongate, flexible and implantable body, which in accordance with one embodiment of the system is preferably formed as an electrode or preferably comprises an electrode, and a plurality of x-ray markers, which are arranged on the body at a distance from one another along said body.

Again, as already explained above, the x-ray markers are preferably annular, more specifically preferably formed as sleeves, wherein adjacent x-ray markers are preferably arranged at distances from one another of 1 cm to 5 cm, preferably 2 cm. All adjacent x-ray markers are preferably arranged at the same distances (for example, D=2 cm). The lengths of the sleeves can vary depending on position. This is also true for the method and computer program described herein.

In accordance with a preferred embodiment of the system according to the present invention, provision is made for the x-ray markers to comprise metal particles, which are introduced in ring form into an insulation of the body, or for the x-ray markers to each comprise a metal braid, which is arranged in an insulation of the body or the electrode (see also above).

In accordance with a preferred embodiment of the system according to the present invention, provision is also made for the x-ray markers to differ from one another in terms of their spatial dimensions, in particular depending on their position along the body, in particular such that the corresponding contrasts of the x-ray markers in the 2D x-ray image can be distinguished from one another (see also above).

A preferred embodiment of the system according to the present invention is also constituted by the fact that the system comprises an x-ray device configured to generate a 2D x-ray image of the body, and also an analysis means configured to determine the two-dimensional positions of the x-ray markers in a 2D image coordinate system of the 2D x-ray image and furthermore configured to determine possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane, and also configured to repeatedly determine the spatial course of the body with use of said possible 3D location coordinates.

The analysis means can comprise a computer (for example, a commercially available PC), on which a suitable software (for example, the computer program according to the present invention) is run or can be run. However, the analysis means can also be formed differently (for example, as a pure hardware solution having fixedly integrated software).

In accordance with a preferred embodiment of the system according to the present invention, provision is also made for the analysis means to be configured, for said repeated determination:
(a) to fit a 3D curve to predefined starting values for the 3D location coordinates from the set of possible 3D location coordinates (for example, on the plane centrally between the radiation source and a detector of the x-ray device parallel to the detector or image coordinate plane (one point in space per x-ray marker)),
(b) to shift a 3D location coordinate of the 3D curve along the associated beam to a possible further 3D location coordinate in order to obtain updated 3D location coordinates,
(c) to fit a 3D curve to the updated 3D location coordinates,
(d) to perform a back-projection of the 3D curve for the updated 3D coordinates into the image coordinate system (onto the 2D image coordinates) and to compare the back-projection with the 2D x-ray image, more specifically in particular with regard to the size, the orientation, and the position of the x-ray markers in the x-ray image, and
(e) to continue the repeated determination starting with step (b) until a predefined criterion is reached.

In accordance with a preferred embodiment of the system according to the present invention, provision is also made for the analysis means to be configured to select the further location coordinate in step (b) such that it lies on the beam associated with the further location coordinate and in a spherical shell around a 3D location coordinate of an adjacent x-ray marker, wherein the outer radius of the spherical shell is given by the distance between the two adjacent x-ray markers along the body, and wherein the inner radius of the spherical shell is given by the length of a chord extended between the two x-ray markers with maximum curvature of the body between the two adjacent x-ray markers (reference is made in this respect to the above explanations).

Further embodiments, features, aspects, objects, advantages, and possible applications of the present invention could be learned from the following description, in combination with the Figures, and the appended claims.

### DESCRIPTION OF THE DRAWINGS

Further features and advantages of the invention will be explained in the description of the drawings of an exemplary embodiment of the present invention with reference to said drawings, in which:

FIG. 1 shows a schematic illustration of a body according to the present invention in the form of an electrode having x-ray markers which are arranged along the body and are distanced from one another;

FIG. 2 shows a schematic illustration of the generation of a 2D x-ray image of the x-ray markers;

FIG. 3 shows a sequence of an embodiment of a method or computer program according to the present invention for 3D reconstruction of an electrode on the basis of a 2D x-ray image;

FIGS. 4A-4D show a schematic illustration of the 3D reconstruction of an electrode course with a method or computer program according to the present invention; and

FIG. 5 shows an illustration of physical restrictions when shifting a 3D location coordinate to a beam of possible 3D location coordinates.

### DETAILED DESCRIPTION

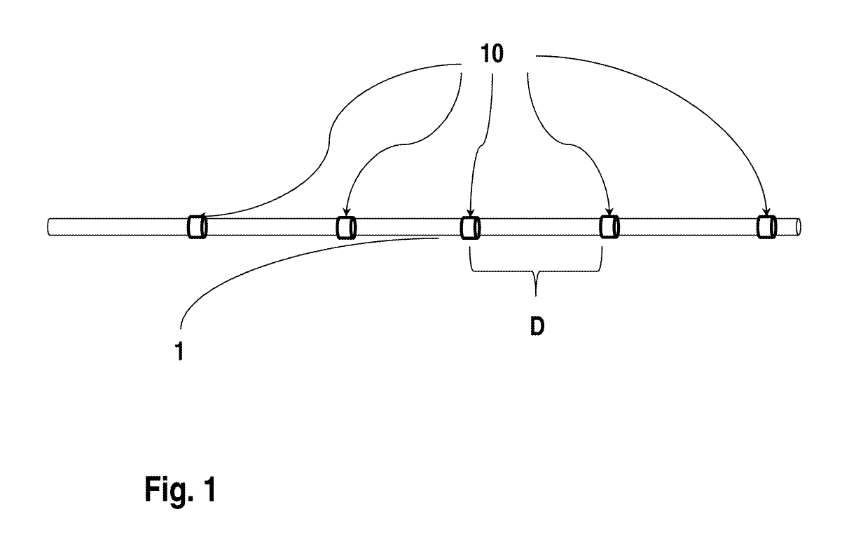

FIG. 1 shows a body 1 according to the present invention in the form of an electrode, in particular a Brady electrode, having an outer diameter of, for example, 2 mm (for example, Siello). The electrode 1 extends along a longitudinal axis and has x-ray markers 10 in the form of metal sleeves (for example, MP35N), wherein adjacent sleeves have a distance from one another of D=2 cm. The sleeves 10, for example, also have an outer diameter of 2 mm and, for example, a length of 1 mm (along the longitudinal axis of the electrode). The distance 2 cm is given, in particular, on the basis of the assumption that the average radii of curvature of electrode 1 in the intracardial field are approximately r=2 cm (curvature: 0.5 l/cm). The wall thickness of the sleeves 10 preferably generates a visible x-ray contrast and, for example, is greater than or equal to 0.05 mm.

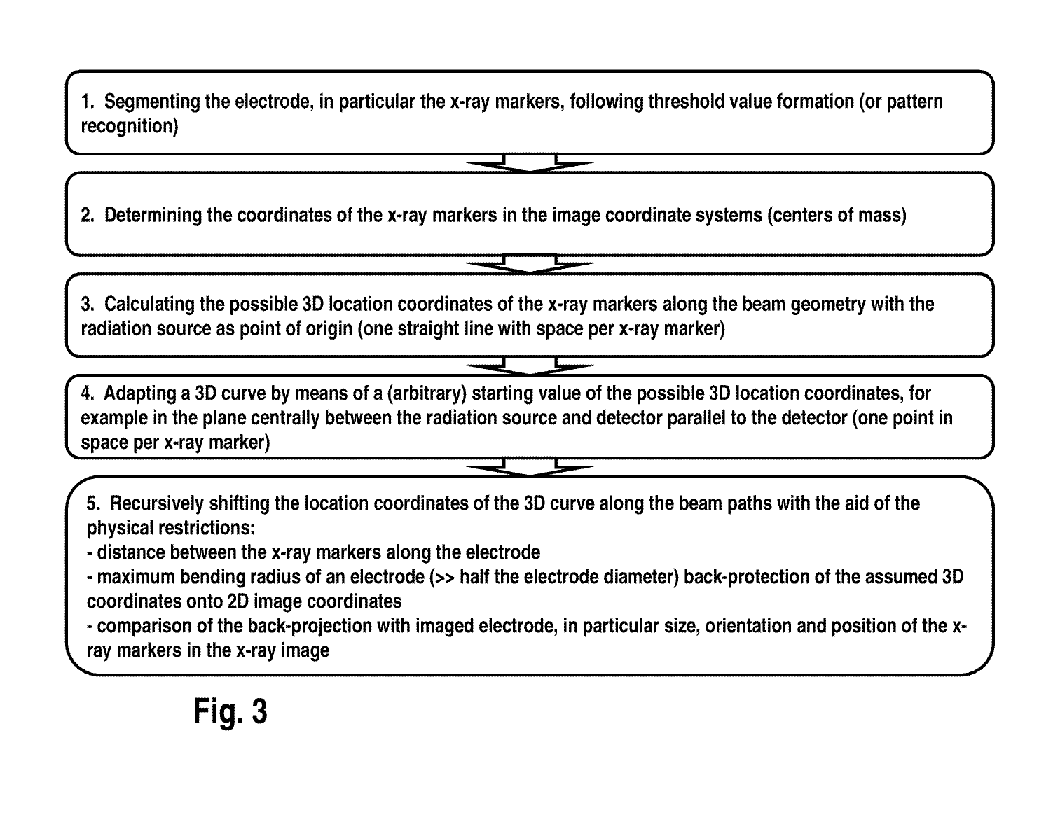

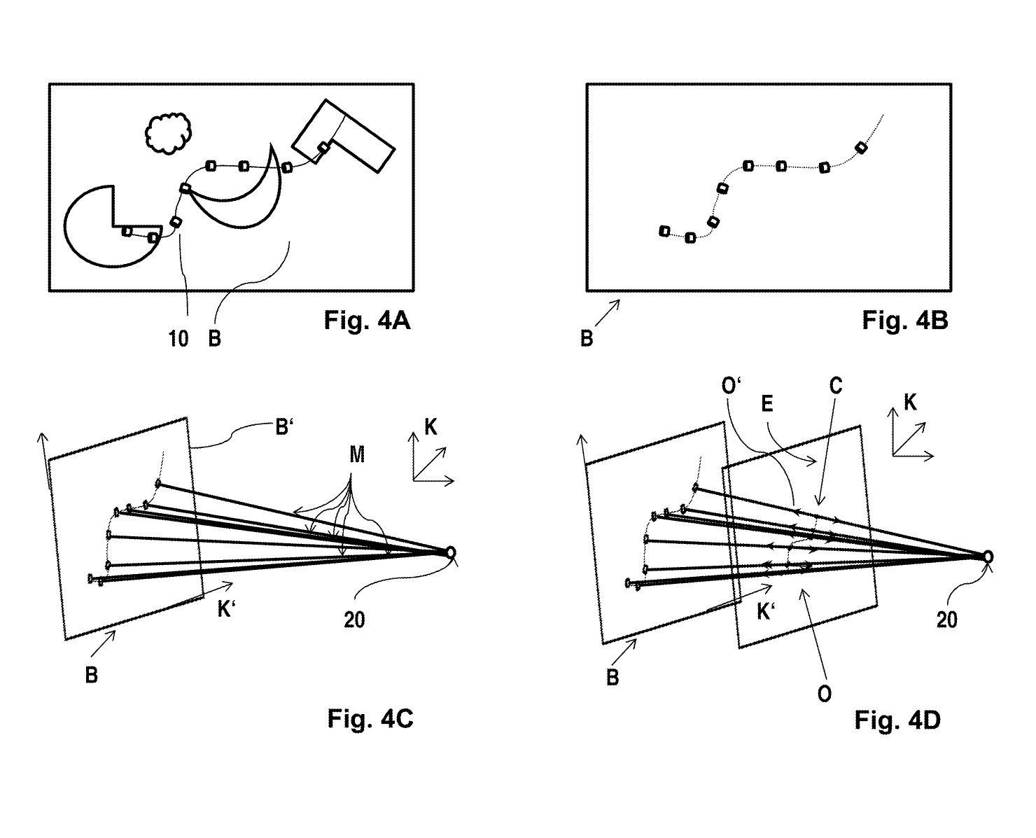

A possible sequence of the 3D reconstruction of the electrode 1 is presented by way of example in FIG. 3. The positions of the sleeves 10 in the 2D x-ray image B (see FIG. 4A) are preferably determined by means of (automatic) pattern recognition (see FIG. 4B). Depending on the x-ray contrast, a conversion of the x-ray image into a binary image following threshold value formation is sufficient for this purpose. The contrast can be increased optionally by the phase contrast and/or edge contrast enhancement (for example, Canny, Sobel or Prewitt filter).

Following a segmentation of the x-ray image B, the position of the x-ray markers 10 can be determined in image coordinates (for example, by determining the centers of mass within the binary image B). The position in image coordinates, following application of the beam geometry, leads to possible positions M of the sleeves 10 in the 3D space K along straight lines or beams M with the position of the radiation source 20 as point of origin (see FIG. 4C).

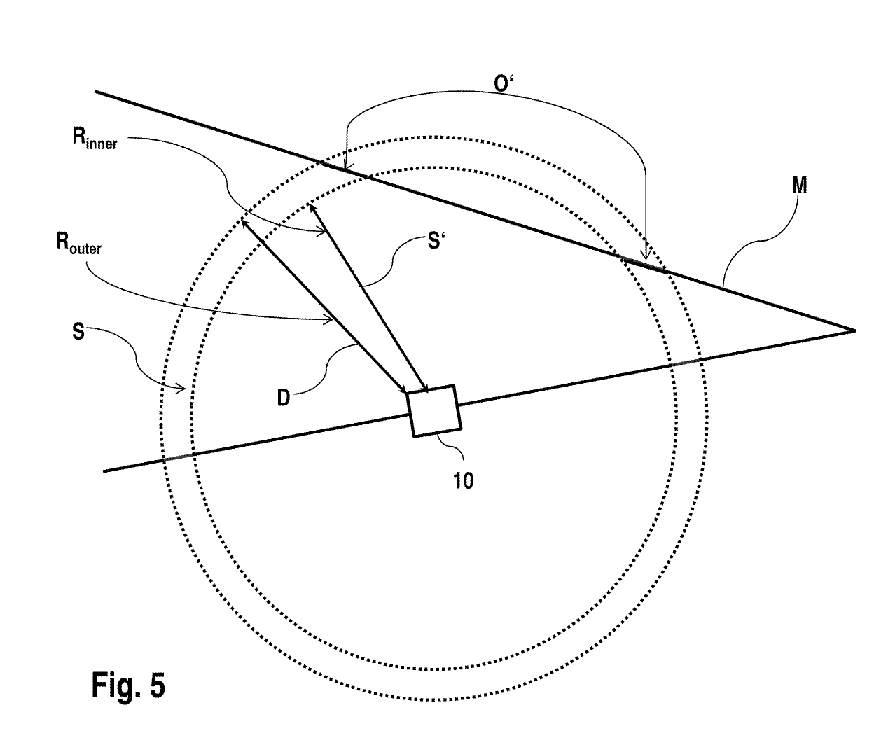

Since the distance between the sleeves 10 is clearly defined and the maximum curvature can be limited on account of the resilient properties and the diameter of an electrode 1, a spherical shell S (see FIG. 5) is given, for each possible position of an n^{th} sleeve 10 on the corresponding straight line M, for the possible position of the adjacent (n+1) sleeve 10.

The outer radius Router of the spherical shell S is equal to the distance D between the adjacent sleeves: Router=D. The inner radius Rinner of the spherical shell S is given here by the length of the chord S′ with maximum curvature r: Rinner=2*r*sin(D/(2*r)).

The straight portions O′, which are given from the intersection of the spherical shell S with the beam M of the adjacent (n+1) sleeve 10, heavily limit the possible 3D positions of the sleeves 10 for the optimization of the electrode position.

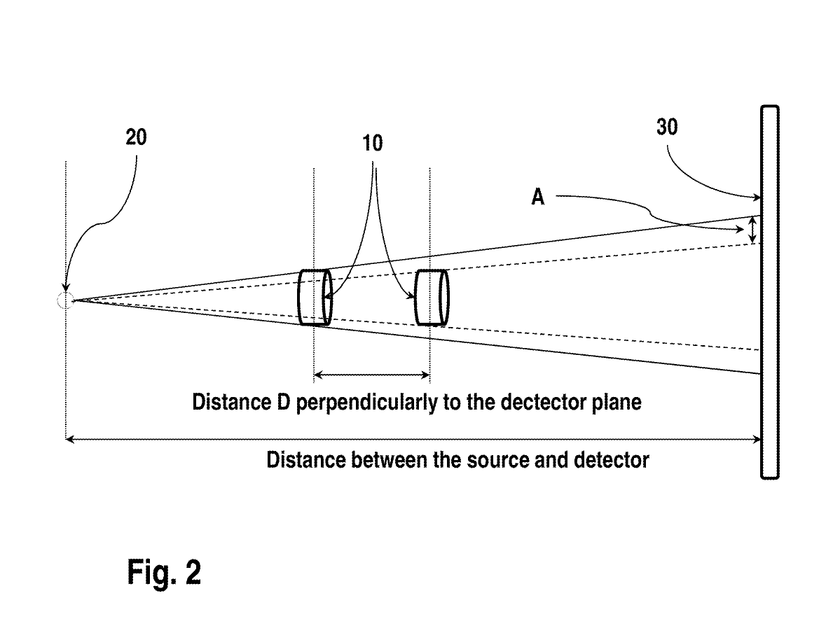

The information as to whether the position of the sleeve 10, starting from the position of the radiation source 20, lies on the front or rear straight-line portion can be determined from the parallactic enlargement of the sleeve (see FIG. 2). Additional information provides the angular position and geometric shortening of the sleeve 10 in the detector plane of the detector 30. The spatial resolution capability outside the image axis is dependent primarily on the resolution capability of the x-ray image B.

With a typical distance of radiation source 20 from radiation detector 30 of 1 m and an assumed distance of the electrode from the radiation source of approximately 0.5 m, the spatial depth resolution with a typical image resolution of the detector 30 of 0.2 mm is approximately 2.6 cm.

The 3D coordinates of the electrode 1 are preferably determined by recursive determination of the 3D location coordinates of the x-ray markers 10 proceeding from a starting value E and curve fitting (for example, non-negative least square or NNLS) by the assumed positions of the sleeves. For the curve fitting, a polynomial of fourth order is preferably used, since the elastic parameters of an electrode 1 can thus be imaged most accurately.

With each repetition step, the positions of the sleeve 10 are shifted along the associated location straight lines M under consideration of the physical restrictions (see above, FIG. 5), a 3D curve C is fitted by the new location coordinates O (see FIG. 4D), and this curve C is projected onto the x-ray image B. Following a comparison between this projection and the actual position of the electrode 1 in the x-ray image B, the location position of the markers 10 is modified accordingly and the most likely position is determined by means of conventional optimization algorithms (for example, gradient methods, particle swarm optimization, genetic algorithm). If the imaging of the sleeves in the back-projection is smaller than the size of the imaging in the 2D x-ray image, the position of the sleeve is shifted in the direction of the radiation source. If the imaging of the sleeves in the back-projection is larger than the size of the imaging in the 2D x-ray image, the position of the sleeve is shifted in the direction of the image coordinate plane.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A method for reconstructing the spatial course of an elongate, flexible body in a 3D world coordinate system, wherein the body has a plurality of x-ray markers, which are arranged on the body distanced from one another along said body, said method comprising the following steps:
providing a 2D x-ray image of the body;
determining the two-dimensional positions of the x-ray markers in an image coordinate system of the 2D x-ray image;
determining possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane; and
repeatedly determining the spatial course of the body with use of said possible 3D location coordinates.

2. The method as claimed in claim 1, wherein the body comprises an electrode or is formed as an electrode.

3. The method as claimed in claim 1, wherein said repeated determination comprises the following steps:
(a) pre-defining starting values for the 3D location coordinates of the x-ray markers in the world coordinate system from the set of possible 3D location coordinates (M) and fitting a 3D curve to said starting values;
(b) shifting at least one 3D location coordinate along the associated beam to a possible further 3D location coordinate in order to obtain updated 3D location coordinates of the x-ray markers in the world coordinate system;
(c) fitting a 3D curve to the updated 3D location coordinates;
(d) back-projecting the 3D curve into the image coordinate system and comparing the back-projection with the 2D x-ray image; and
(e) continuing the repeated determination starting with step (b) until a predefined criterion is reached.

4. The method as claimed in claim 3, wherein, in step (b), the further location coordinate is selected such that it lies on the beam associated with the further location coordinate and in a spherical shell around a 3D location coordinate of an adjacent x-ray marker, wherein the outer radius (Router) of the spherical shell is given by the distance between the two adjacent x-ray markers along the body, and wherein the inner radius (Rinner) of the spherical shell is given by the length of a chord extended between the two x-ray markers with maximum curvature of the body between the two adjacent x-ray markers.

5. The method as claimed in claim 1, wherein the x-ray markers are annular and are formed as sleeves.

6. A computer program for reconstructing the spatial course of an elongate flexible body in a 3D world coordinate system, wherein the body has a plurality of x-ray markers, which are arranged on the body distanced from one another along said body, and wherein the computer program comprises a program code, which is configured to perform the following steps when the computer program is run on a computer:
determining the two-dimensional positions of the x-ray markers in an image coordinate system of a 2D x-ray image recorded by the body;
determining possible 3D location coordinates of each x-ray marker in the 3D world coordinate system as beams each extending from a point of origin, which corresponds to the position of the radiation source for generation of the 2D x-ray image, to the position of the x-ray marker in question in an image coordinate plane; and
repeatedly determining the spatial course of the body with use of said possible 3D location coordinates.

7. The computer program as claimed in claim 6, wherein said repeated determination comprises the following steps:
(a) fitting a 3D curve to predefined starting values for the 3D location coordinates of the x-ray markers in the world coordinate system from the set of possible 3D location coordinates;
(b) shifting at least one 3D location coordinate along the associated beam to a possible further 3D location coordinate in order to obtain updated 3D location coordinates of the x-ray markers in the world coordinate system;
(c) fitting a 3D curve to the updated 3D location coordinates;
(d) back-projecting the 3D curve into the image coordinate system and comparing the back-projection with the 2D x-ray image; and
(e) continuing the repeated determination starting with step until a predefined criterion is reached.

8. The computer program as claimed in claim 7, wherein, in step (b), the further location coordinate is selected such that it lies on the beam associated with the further location coordinate and in a spherical shell around a 3D location coordinate of an adjacent x-ray marker, wherein the outer radius (Router) of the spherical shell is given by the distance between the two adjacent x-ray markers along the body, and wherein the inner radius (Rinner) of the spherical shell is given by the length of a chord extended between the two x-ray markers with maximum curvature of the body between the two adjacent x-ray markers.

9. A system for reconstructing the spatial course of an elongate, flexible body in a 3D world coordinate system, comprising:
an elongate, flexible and implantable body, which is formed as an electrode or comprises an electrode; and
a plurality of x-ray markers, which are arranged on the body distanced from one another along said body.

10. The system as claimed in claim 9, wherein the x-ray markers are annular and are formed as sleeves.

11. The system as claimed in claim 9, wherein the x-ray markers comprise metal particles, which are introduced in ring form into an insulation of the body, or in that the x-ray markers each comprise a metal braid, which is arranged in an insulation of the body.

12. The system as claimed in claim 9, wherein the x-ray markers differ from one another in terms of their spatial dimensions.

13. The system as claimed in claim 9, wherein the system also has an x-ray device configured to generate a 2D x-ray image of the body, and an analysis means configured to determine the two-dimensional positions of the x-ray markers in an image coordinate system of the 2D x-ray image, and further configured to determine possible 3D location coordinates of each x-ray marker in the 3D world coordinate system as beams each extending from a point of origin, which corresponds to the position of a radiation source of the x-ray device for generation of the 2D x-ray image, to the position of the x-ray marker in question in an image coordinate plane, and further configured to repeatedly determine the spatial course of the body with use of said possible 3D location coordinates.

14. The system as claimed in claim 13, wherein the analysis means is also configured, for said repeated determination:
(a) to fit a 3D curve to predefined starting values for the 3D location coordinates from the set of possible 3D location coordinates;
(b) to obtain updated 3D location coordinates, to shift at least one 3D location coordinate of the 3D curve along the associated beam to a possible further 3D location coordinate in order to obtain updated 3D location coordinates;
(c) to fit a 3D curve to the updated 3D location coordinates;
(d) to perform a back-projection of the 3D curve into the image coordinate system and to compare the back-projection with the 2D x-ray image; and
(e) to continue the repeated determination starting with step until a predefined criterion is reached.

15. The system as claimed in claim 14, wherein the analysis means is configured to select the further location coordinate in step (b) such that it lies on the beam associated with the further location coordinate and in a spherical shell around a 3D location coordinate of an adjacent x-ray marker, wherein the outer radius (Router) of the spherical shell is given by the distance between the two adjacent x-ray markers along the body, and wherein the inner radius (Rinner) of the spherical shell is given by the length of a chord extended between the two x-ray markers with maximum curvature of the body between the two adjacent x-ray markers.
